(19) United States
(12) Patent Application Publication
(10) Pub. No.: US 2026/0157115 A1
(43) Pub. Date: Jun. 4, 2026

(54) PACKAGING AND MANUFACTURE OF MAGNETOELECTRIC FILMS FOR MEDICAL DEVICES AND DEVICES PRODUCED THEREFROM

(71) Applicant: Motif Neurotech, Inc., Houston, TX (US)

(72) Inventors: Amanda Singer, Houston, TX (US); Jia Hu, Houston, TX (US); Elizabeth Commissaris, Houston, TX (US); Steven Goetz, Houston, TX (US); Phil Meneau, Houston, TX (US); Landan Mintch, Houston, TX (US)

(73) Assignee: Motif Neurotech, Inc., Houston, TX (US)

(21) Appl. No.: 19/372,054

(22) Filed: Oct. 28, 2025

Related U.S. Application Data
(60) Provisional application No. 63/713,992, filed on Oct. 30, 2024.

(51) Int. Cl. H10N 39/00; A61N 1/378

(52) U.S. Cl. CPC H10N 39/00; A61N 1/3787

(57) ABSTRACT

The present disclosure relates to improving the robustness and reliability of magnetoelectric (ME) films by fine-tuning different parameters of the films and providing additional features for operation of the ME films for long periods of time. The additional features may include e.g. mechanical holding such as a hermetic enclosure, electrical connections, hermetic protection, while parameters of the ME film may include geometric configurations including shape and the point of placement. Other additional features may include incorporating additional elements such as microelectronics into the hermetic enclosure as a system on module, adding desiccant elements, and bias magnets to enhance strain on the ME films. The ME films can also be miniaturized to make them suitable for integration into small-scale devices due to their compact size, low power consumption, and a high sensitivity to electric and magnetic fields.

### CROSS-REFERENCE OF RELATED APPLICATIONS

This application claims priority to U.S. Provisional Ser. No. 63/713,992, filed on Oct. 30, 2024, titled “PACKAGING AND MANUFACTURE OF MAGNETOELECTRIC FILMS FOR MEDICAL DEVICES AND DEVICES PRODUCED THEREFROM”. The entire disclosure of the aforementioned application is incorporated by reference herein in its entirety for all purposes.

### BACKGROUND

Magnetoelectric materials are characterized by their ability to convert magnetic energy into electrical energy and vice versa, making them suitable for various technological applications including medical devices, wireless power transfer, Internet of Things (IoT), aerospace applications, environmental sensors and energy harvesting solutions. In recent years, magnetoelectric (ME) films have gained significant attention due to their ability to enable compact, efficient, and miniaturized designs, typically with lateral dimensions around 1 cm while maintaining a much smaller thickness in the micrometer or nanometer range. ME films may be particularly useful in miniaturized designs because ME films are sensitive to magnetic and electric fields, as they can sense magnetic fields of the order of nanotesla (nT) and electric fields of the order of microvolts (μV). The miniaturized design may be used across a spectrum of fields including microsensors, micro-actuators, and components for micromechanical systems (MEMS). The ME films can self-power themselves by utilizing energy harvesting techniques to manufacture compact, efficient, and reliable devices. Some examples include miniaturized medical implants that may be implanted inside or on a human body for biosensing, targeted drug delivery, and neural. The medical devices, using ME films, may require lesser maintenance (e.g., wearable health monitors or implantable medical devices can harvest energy from mechanical movements or external magnetic fields, thus reducing the frequency of battery replacements).

However, the performance of magnetoelectric (ME) films may be compromised without appropriate design and protective measures. For example, improper design, such as spacing issues may hinder resonance of the ME film and inclusion of additional components may disrupt electrical pathways in ME films. Moreover, ME based devices may experience overheating due to low dissipation capabilities of ME films during generation and manipulation of magnetic fields. Inadequate support may lead to physical damage during handling or operation, undermining the durability of the films, while improper integration of support structures or connections may create interference in the resonance of the ME films, leading to fluctuations in performance and reducing overall efficiency. Additionally, exposure to environmental factors may result in degradation, which diminishes functionality and shortens lifespan. The lack of effective strategies for maintaining stability may increase the risk of operational failure, particularly in miniaturized applications with limited space. Without suitable protective measures, the ME films may become vulnerable to contaminants, further exacerbating performance issues. These challenges may highlight significant obstacles in achieving reliable and effective applications for magnetoelectric films.

### SUMMARY

Certain aspects of the present disclosure relate to techniques to improve packaging and apparatus for magnetoelectric (ME) films by adjusting one or more parameters of ME films, such as geometry and providing additional features e.g., electrical connections and mechanical holding (e.g., a hermetic enclosure and/or one or more retention features) for miniaturized ME films. One or more ME films may be comprising a magnetostrictive layer that comprises a magnetostrictive element configured to be magnetized, inducing mechanical strains when an external magnetic field is applied. Each magnetostrictive layer may include a piezoelectric layer that comprises a piezoelectric element configured to generate an electrical signal in response to mechanical strains from at least one layer of magnetostrictive material. To mechanically hold one or more ME films, the hermetic enclosure may be used to enclose the one or more ME films with a spacing around the ME films to allow resonance of the ME films. The spacing or adjacency metric may vary with the film size; for instance, a spacing of approximately 5 mm between miniaturized films of 6 mm height by 2 mm width might be fixed to improve resonance. The hermetic enclosure may be configured to mechanically hold the one or more ME films by including one or more retention features.

The one or more retention features may include an inward curving geometry of any two parallel sides of the hermetic enclosure to hold the one or more ME films from the any two parallel sides. The one or more retention features may further include one or more pins configured to pierce through the one or more ME films, and the one or more pins can be of different shapes and sizes configured to fix the one or more ME films by pressing it. The shape of one or both ends of the one or more pins may be flat or curved such that their contact region with the one or more ME films does not exceed 5 to 15 percent of the total length of the one or more ME films.

To enable free resonance in the one or more ME films, positioned inside the hermetic enclosure, each of the one or more ME films may be placed with a fixed spacing in between two adjacent ME films such that the spacing may be filled by the electrical connections e.g., a plurality of metallized pads, microwires or other connective media.

In some aspects, an apparatus may include the one or more ME films, having trapezium shapes that are joined together from each parallel side of smaller length within the hermetic enclosure. The resulting one or more ME films may be held or positioned inside the hermetic enclosure using the one or more pins positioned along a longitudinal axis to reduce the mechanical damping of the one or more ME films at resonance.

The electrical connections to the one or more magnetoelectric films, enclosed within the hermetic enclosure, may be enabled through a plurality of metallized pads and through-glass vias (TGVs). The plurality of metallized pads may be configured to connect each adjacent film of the one or more magnetoelectric films, whereas through-glass vias (TGVs) may be designed to penetrate the hermetic enclosure, connecting the one or more magnetoelectric films from both sides to external electronics, via the metallized pads. The apparatus may further comprise one or more bias magnets that can be positioned in proximity to the one or more magnetoelectric films. The bias magnets may be positioned to enhance the mechanical strains that are induced on the at least one layer of magnetostrictive material. The material of the hermetic enclosure can be glass, ceramic or epoxy.

In some other examples, the apparatus of the one or more ME films may include a carrier board, a partial hermetic cap that encloses the one or more ME films with a spacing around each of the one or more magnetoelectric film to allow resonance such that the one or more ME films can be directly positioned on the carrier board. Since the largest element in terms of length in a medical device may be the ME films, therefore, the films may be used as a substrate for assembling wafer films or system on a module. In some examples, the plurality of microelectronics can be positioned directly on either side of the one or more ME films such that they may be used as a substrate to enable the assembly of microelectronics on the wafer film on the carrier board.

The partial hermetic cap that may be used to enclose the one or more magnetoeletric films and the microelectronics on the wafer film may include processes to enable electrical connection such as connectors, pins through hole assembly, metallized pads, through glass vias (TGV), etc. The apparatus of the one or more ME films may further comprise of one or more bias magnets that can be positioned in proximity to the one or more magnetoelectric films.

In some embodiments, a system is provided that includes one or more data processors and a non-transitory computer readable storage medium containing instruction which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows an example illustration of medical implants utilizing one or more magnetoelectric (ME) films enclosed in a hermetic enclosure for one or more medical implants.

FIG. 2 shows an example illustration of an equivalent circuit model of an ME film when it is subjected to an external alternating magnetic field.

FIG. 3 shows an example illustration of a hermetic enclosure for packaging of the one or more ME films to enhance electrical and mechanical holding.

FIG. 4 illustrates one or more exemplary placements, and geometry of one or more pins to hold the one or more ME films inside hermetic enclosure.

FIG. 5 shows exemplary illustrations of varying shapes of the one or more pins to hold the one or ME films inside the hermetic enclosure.

FIG. 6 shows an example illustration for placing the one or more ME films inside the hermetic enclosure by incorporating one or more additional features for electrical connections.

FIG. 7 shows exemplary arrangements of a plurality of microelectronics, packaged with the one or more ME films, positioned on a carrier board capped with a partial hermetic cap.

FIG. 8 shows an example illustration of an exploded view of the hermetic enclosure in accordance with some embodiments of the present disclosure.

### DETAILED DESCRIPTION

Some embodiments of the present disclosure relate to improving the robustness and reliability of magnetoelectric (ME) films by fine-tuning various parameters of the ME films and providing additional features for the film, interchangeably used with ME film. These parameters may include e.g. shape and size that may be adjusted by modifying geometry of the ME film, while additional features may include e.g., electrical connections and/or mechanical holding, such as hermetic protection. Other additional features may also include incorporating various components, such as microelectronics added to the enclosure as a system-on-module, and strain enhancer elements such as desiccants, and bias magnets. The ME films can be miniaturized because of their compact size and low power consumption (e.g., in microwatts (μW) or milliwatts (mW)), making them suitable for integration into small-scale devices, and yet remain sensitive to electric and magnetic fields in their microenvironments.

The ME films may comprise of a layered structure having one or more layers of piezoelectric material (e.g., lead zirconate titanate (PZT), polyvinylidene fluoride (PVDF), or composites of these material) that may be deposited on one or more layers of magnetostrictive material having high permeability (e.g., iron, nickel, boron or their alloys such as Metglas, Terfenol-D. Under the influence of the magnetic field, the magnetostrictive elements may be magnetized, resulting in the induction of mechanical strains within the magnetostrictive material. These mechanical strains may be transferred to the adjacent piezoelectric layer that may eventually generate an electrical response. Magnetostrictive materials, which can be sensitive to environmental conditions like humidity and water, may degrade or corrode when exposed to moisture in the environment. It may be an effective approach to protect the entire device or the medical implant rather than the magnetostrictive material only.

The additional features such as packaging, electrical connections, and mechanical holding of the magnetoelectric (ME) films may be improved by modifying the shape (contour or geometry) of the ME films in various ways. For example, one or more ME films may be protected by putting them inside a hermetic enclosure. The hermetic enclosure or a near-hermetic enclosure may be a sealed environment designed to protect electronic components, sensors, or other sensitive materials from environmental conditions such as moisture, dust, gases, and contaminants. Near-hermetic enclosures may offer similar protection but may allow minimal permeation of gases or moisture over time. These enclosures may be used in applications working in controlled environments, such as in the domains of aerospace, medical devices, and high-reliability electronics.

Two different approaches may be applied to achieve hermetic protection for a medical device. In the first approach, the box itself can be made to act like a hermetic seal. This could involve using materials like glass micro-enclosures with hermetic vias (small channels that maintain a seal while allowing electrical connections) and laser-welded edges to create a seal that protects the device from the environment. Alternatively, or additionally, a ceramic circuit board may be used in which different sections may be soldered together to form an airtight enclosure, protecting exposure of sensitive internal components to moisture or gases. In the second approach, the enclosure may serve as a barrier that may not be inherently fully hermetic, but it may still provide protection by packaging the device in a polymer casing. The medical device inside the enclosure may then be filled with materials like epoxy, silicone, or other polymers to reduce the rate of seep of water or moisture into the device

Additionally, the ME films embedded inside the hermetic enclosure may need space within an appropriate hermetic enclosure to resonate freely. Free and undamped resonance may be enabled if the ME films are detached from the internal circuitry, inside the medical implants, and are fixed using minimum contacts in a mechanical holding while having electrical connections. Separating mechanical holding of electrical connections in the ME films may enable free resonance, providing flexibility in the design and manufacturing processes, thereby improving mechanical structure and the electrical performance of a medical implant.

In some aspects of the present disclosure, the separation between electrical connections and mechanical holding may be achieved by using one or more retention features that involve cutting features into the ME film. These features, such as slots, notches, or holes, can be pierced in the ME films to precisely place and hold the films within the hermetic enclosure. Cut-out sections may not interfere with the electrical properties of the ME films but may still mechanically secure the ME films—using clamps, one or more pins, or other holding components—at the anchoring points without the electrical contacts for the support. The retention features may also reduce the risk of damaging the electrical connections during assembly process at the plant or operations in the field.

In some examples, the contact points of the pins may have linear or curved shapes. The specific point of contact between a pin and an ME film may have different geometrical configurations, improving the mechanical holding of the ME film by reducing stress concentrations. The pin contact points touch the ME film at a precise location, such as the center of the film or any other region, exhibiting the least stress at the contact points when excited. The precise contact points also avoid introducing the excessive damping that could negatively impact the resonance behavior of the ME films. The pins may be rectangular or cross-shaped, determined based on the stress or strain concentrations present in the ME films, when subjected to excitations in an external magnetic field.

Additionally, an inward curved geometry of the hermetic enclosure can help in holding, aligning, or fixing the one or more ME films. By applying a compressive force to the ME films from the sides, the inward curved geometry may be configured to offer mechanical retention, providing stability from inside the hermetic enclosure and reducing resonant damping. The edges of the ME films can be positioned in a recess or channel created by the parallel sides of the inward curved geometry of the hermetic enclosure. The functional region of the ME films may remain unaffected by the curvature, which applies pressure along the length of the edges of the ME film to enable a secure fit.

External and internal connections to the ME films, positioned inside the hermetic enclosure, may be enabled by incorporating one or more additional features for electrical connections. The electrical connections to the ME films can be made through a plurality of conductive elements such as metallized pads, through-glass-vias (TGVs), microwires or other connective media that is traditionally placed on the exterior surfaces of the films.

In some examples, where only one ME film is utilized, the metallized pads may be positioned between adjacent magnetostrictive and piezoelectric layers that form one single ME film. In some other examples, where a plurality of ME films are used, the metallized pads may connect adjacent ME films and adjacent magnetostrictive and piezoelectric layers.

In some aspects, a plurality of microelectronics that are positioned on a carrier board covered with a partial hermetic cap, may be positioed on the ME films. The ME films may be employed as a substrate for the microelectronics, where the microelectronics may be on a thin film structure and assembled on the carrier board, forming an integrated system such as a system-on-chip or system-on-module. The partial hermetic cap may be configured to cover both the ME films and the microelectronics, providing protection from the environment and allowing for electrical connectivity. Additionally, the metallized pads and TGVs may also be incorporated for enabling electrical connections between the ME films and external electronics within a medical implant.

The carrier board may include a circuit board, also referred to as a breakout board or adapter board, designed to host and interface with microelectronics, smaller electronic modules, chips, or components. The carrier board, that serves as a structural base, may be configured to place the ME films and the microelectronics on it, while providing the electrical connections for integration into larger systems or medical implants.

In some other aspects, the hermetic enclosure and the partial hermetic cap may have bias magnets at fixed locations. The bias magnets may be positioned near or relative to the position of the ME films.

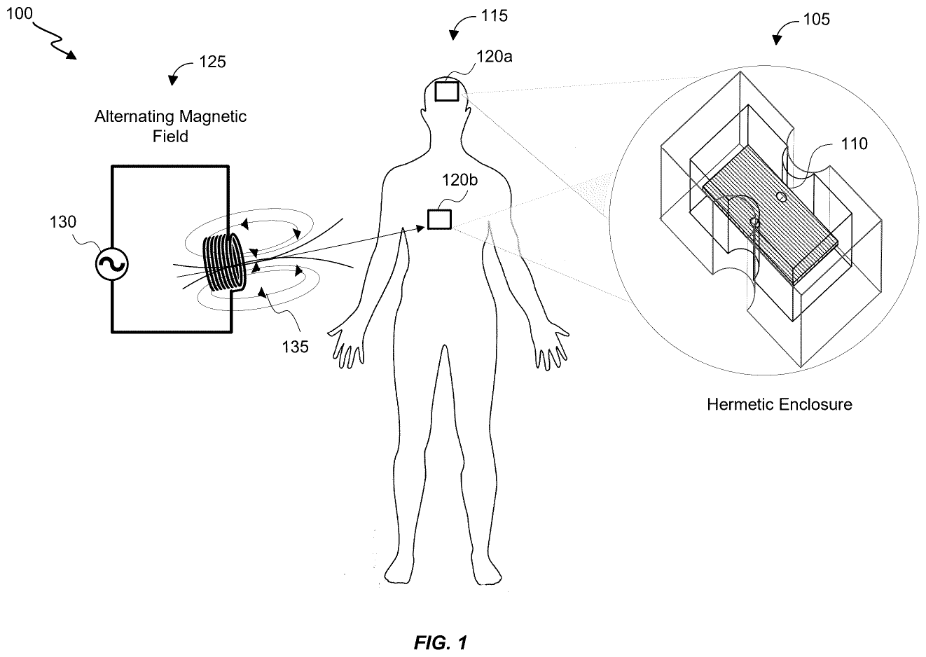

FIG. 1 shows an example illustration of the medical implants utilizing one or more magnetoelectric (ME) films enclosed in the hermetic enclosure. ME materials may be leveraged to harvest energy from ambient sources—such as vibrations, mechanical movements, and magnetic fields—which can power low-power electronic devices in remote or inaccessible locations. ME-enabled medical implants can be used in diverse applications: ME materials may help in wireless charging of electronic gadgets (e.g., smartphones, smartwatches, and other portable devices), industrial applications (e.g., robots and electric vehicles), Internet of Things (IoT), and smart cities, where wireless sensors are deployed for autonomous environmental monitoring or infrastructure management. Moreover, some ME materials are made to be biocompatible and responsive to external stimuli, therefore they are used in biomedical applications, such as therapeutic ultrasound, drug delivery systems, tissue engineering (i.e., for developing smart biomaterials to regenerate tissues and neural interfaces). ME based ultrasound devices can also generate acoustic waves for non-invasive medical imaging and therapeutic treatments. ME materials can help in developing precision control for drug delivery mechanisms, utilizing the magnetic and electric fields, to significantly enhance their performance. Hermetic enclosure 105 provides a hermetic protection to the ME films, minimizes water and moisture seepage to the enclosure - a necessary design requirement for biomedical implants.

Devices (e.g., medical implants) 120a and 120b within or on a human body 115 may require robust and appropriate microenvironments, for a reliable and continuous functioning, to avoid invasive procedures for replacing medical implants periodically. One or more ME films 110, which may combine magnetostrictive and piezoelectric properties, can efficiently convert an alternating magnetic field 125 into electrical energy 240, thereby providing a sustainable power supply for medical devices (e.g., pacemakers, insulin pumps, and neural stimulators). The use of wireless power transfer mechanisms with high power transferring efficiency may improve patient's quality of life, as the need for frequent surgeries to replace batteries in medical devices is eliminated, resulting in reliable medical devices that provide uninterrupted services. For powering the medical implants 120a and 120b, an external transmitter coil 135, connected to a voltage source 130, may generate the alternating magnetic field 125. The one or more ME films 110, embedded in the medical implants 120a and 120b and placed inside a hermetic enclosure 105, may interact with the alternating magnetic field 125 as it penetrates through the human body 115. The alternating magnetic field 125 may induce a change in the magnetization of the magnetostrictive material, resulting in strains that can mechanically deform the material. As a result, an electric field with a corresponding voltage may be induced in an adjacent piezoelectric layer. This voltage source can then be used (e.g., via electrodes) to power the medical implants 120a and 120b or charge their internal batteries, eliminating the need for providing physical connections or replacing internal batteries periodically. In some examples, the generated electric field from the piezoelectric layer may further interact wirelessly with another device in its ambient environment.

The size of hermetic enclosure 105 is kept small to enable miniaturization of medical devices so that they can be implanted in human body 115 using minimal invasive surgery procedure. The one or more ME films 110 are suitable for this use case because they can provide high voltage output even when they are miniaturized. But important factors—such as the geometry of the magnetoelectric film including its height (or ratio of height to width), thickness, orientation relative to incident fields, and spacing—may still need to be optimized to allow for efficient wireless power transfer.

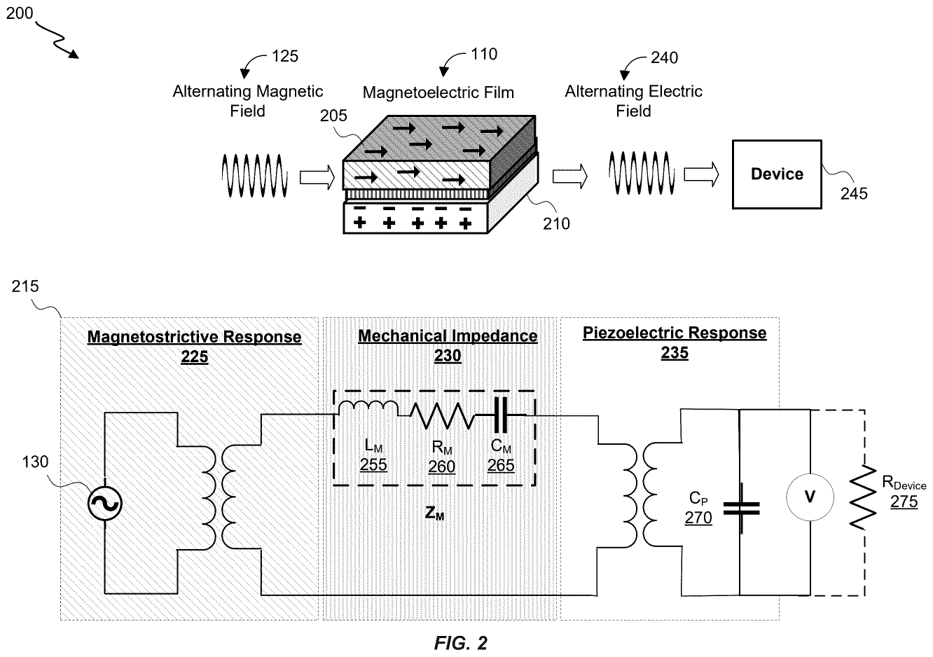

FIG. 2 shows an example illustration of an equivalent circuit model of a magnetoelectric (ME) film when it is placed in the vicinity of an external alternating magnetic field. The working principle of the one or more ME films 110 is the magnetoelectric coupling, where the alternating magnetic field 125 induces an electrical response and vice versa. The geometry of the one or more ME films 110 typically includes a layered structure, combining one or more layers of magnetostrictive layer 205 and one or more layers of piezoelectric layer 210. This composition may generate magnetoelectric effect when the external alternating magnetic field 125 is applied. The magnetostrictive layer 205 is typically made of metallic materials with a high permeability such as iron, nickel, boron or their alloys (e.g., Metglas or Terfenol-D, which exhibit changes in their geometry in response to the applied magnetic field 125. Moreover, the magnetostrictive layer 205 may also experience changes in mechanical strains because of interactions with the alternating magnetic field 125. This strain can induce a mechanical stress within the magnetostrictive layer 205 that may be transferred to an adjacent piezoelectric layer, comprising of materials such as lead zirconate titanate (PZT), polyvinylidene fluoride (PVDF), or composites of these materials. In response, piezoelectric layer 210 may generate an alternating electric field 240 and a corresponding voltage (e.g., across electrodes attached to piezoelectric layer). The amplitude of the voltage can be modulated by shifting the frequency of the applied alternating magnetic field 125.

The one or more ME films 110, other than the magnetostrictive layer 205 and the piezoelectric layer 210, may also include additional layers or components to meet the specific design requirements of applications. These additional layers or components can provide protective coatings or may help in enhancing the structural integrity and the electrical conductivity of the one or more ME films 110. For example, a substrate layer can provide a foundation on which the magnetostrictive layer 205 and the piezoelectric layer 210 may be etched. The substrate layer can also provide mechanical support for a better stability, and electrical isolation. Moreover, the one or more ME films 110 may include electrodes that are made of conductive materials such as metals (e.g., gold, silver) or conductive polymers. These electrodes may be attached to the one or more ME films 110 for applying electric signals to the piezoelectric layer 210 or extracting electric signals that are generated because of the piezoelectric effect. Additionally, buffer layers, comprising of thin conductive layers or insulating layers, may be used to optimize the interference between different materials within the one or more ME films 110 to reduce stress or prevent diffusion between layers. In some applications, the one or more ME films 110 may be coated with protective layers or located inside an IC (integrated circuit) capsule to protect them from environmental factors (e.g., moisture, corrosion) or make them biocompatible in medical devices.

In the equivalent circuit model of the one or more ME films 110, the voltage source 130 may determine the amplitude of the applied alternating magnetic field 125, generated by an external transmitter coil that may be connected to the voltage source 130. The voltage source 130 may induce an elastic excitation or a mechanical stress (used interchangeably herein) in the magnetostrictive layer 205 with the help of a magnetostrictive response 225, which may depend on the thickness of the magnetostrictive layer 205. Losses in the elastic excitation may be represented by an equivalent Mechanical Impedance (Z_{M}) 230 (alternating current (AC) equivalence of resistance R_{M}260, inductance L_{M}255, and capacitance C_{M} 265), which may depend on the interface adhesion, the mechanical quality factor, the total thickness and the thickness ratio of the one or more ME films 110. The interface adhesion refers to the bonding strength between two adjacent layers (e.g., magnetostrictive layer 205 and piezoelectric layer 210). A stronger interface adhesion may result in an efficient and effective transfer of mechanical strains and electrical signals between layers, thereby increasing power efficiency by reducing power losses.

The mechanical quality factor of the one or more ME films 110 quantifies the efficiency of a mechanical energy storage and dissipation system. It is a measure of how well a mechanical system or material stores and releases energy during vibrations or oscillations, and it may be influenced by factors such as material composition, damping mechanisms (including interface adhesion), and the mechanical design of the one or more ME films 110. A higher mechanical quality factor represents a small energy loss and hence an efficient mechanical response; while in comparison, a lower mechanical quality factor represents a large energy dissipation and damping. Similarly, the thickness ratio refers to the relative thicknesses of the layers within the structure of ME films, such as the magnetostrictive layer 205 and the piezoelectric layer 210. The thickness ratio can impact the mechanical impedance Z_{M} 230 that is determined by parameters including resonant frequencies, mechanical strain distribution, and energy conversion efficiency. A strong mechanical resonance is achieved by adjusting or fine tuning the thickness ratio of ME films, which provides better a coupling between magnetic and electrical responses, thereby improving transduction capabilities to efficiently harvest energy.

The interface coupling factor relates to the interface adhesion and refers to the degree of coupling. The interface coupling factor can be influenced by the packaging technique. In comparison, the mechanical quality factor can be affected not only by these factors but also with the factors like clamping losses due to the damping mechanisms. The elastic excitation may be converted into an electric field 240 with the help of a piezoelectric response 235, where Cp 270 represents the capacitance of the piezoelectric layer 210. The piezoelectric response 235 may also depend on the thickness of the piezoelectric layer 210. The resulting electric field 240 can be used to power a device 245 (e.g., medical implants) wirelessly. In this equivalent circuit model 215 the voltage difference across the one or more ME films 110 may be represented by V and the device resistance by R_{Device} 275

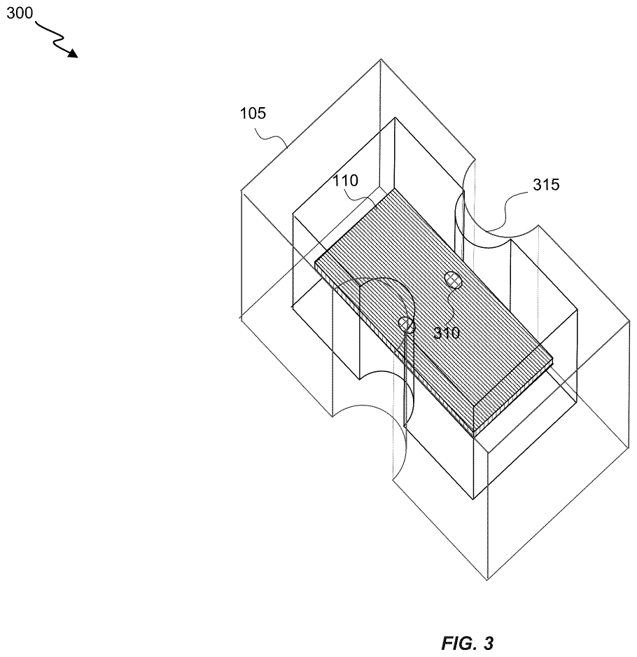

FIG. 3 shows an example illustration of the hermetic enclosure 105 for packaging the one or more ME films 110 to enhance electrical connections and mechanical holding. The hermetic enclosure 105 can be made of glass, ceramic, epoxy or other hermetic material. Moreover, the hermetic enclosure 105 can also use glass micro-enclosures with hermetic vias and laser-welded edges to create a fully sealed environment to protect the one or more ME films 110 from external environmental elements such as moisture, dust, gases, and other contaminants. A near-hermetic enclosure offers a somewhat similar protection but cannot stop little permeation of gases or seepage of moisture over longer periods of time. These enclosures are a necessary requirement for aerospace systems, medical devices, and high reliability electronics, where a strict control of the operating environment control is a design requirement.

In some embodiments, the hermetic enclosure 105 may not be inherently hermetic but still provides protection by encasing the one or more ME films 110 in a polymer material. The medical device inside a box may then be filled with materials like epoxy, silicone, or other polymers that effectively reduce the rate of seepage of water or moisture, thereby extending the operational life of the one or more ME films 110 in humid environments.

In addition to providing a protective covering for the one or more ME films 110, the hermetic enclosure 105 may also include the plurality of film retention features. The film retention features may include the one or more pins 310 or the inward curved geometry 315 of the hermetic enclosure 105. The inward curved geometry 315 of the hermetic enclosure 105 can help in holding, aligning, or fixing the one or more ME films 110. One way may be to use cutting features into the one or more ME films 110 that may allow for mechanical holding inside the hermetic enclosure 105. The geometrical shape of the one or more ME films 110 can vary from regular rectangle films to trapezoidal shaped films.

In some embodiments, the inward curved geometry 315 along any two parallel sides of the hermetic enclosure 105 may be configured to hold the one or more ME films 110 from the parallel sides. The inward curved geometry 315 may be designed to provide mechanical retention by exerting a compressive force on the one or more ME films 110 from the sides, minimizing the damping resonance and ensuring the mechanical stability within the hermetic enclosure 105. The inward curved geometry 315 of the parallel sides of the hermetic enclosure 105 creates a recess or channel in which the edges of the one or more ME films 110 may be positioned. The curvature can be configured to securely maintain the positions of the ME films, applying pressure along the edges (on the length) of the ME films without interfering with the functional area of the one or more ME films 110. This retention mechanism keeps the one or more ME films 110 properly aligned and stable, while minimizing mechanical damping effects that could impact the resonance characteristics of the magnetoelectric material.

Additionally, the inward curved geometry 315 of the hermetic enclosure 105 may be optimized to conform to the specific dimensions of the one or more ME films 110, providing sufficient contact to hold the ME films securely at a place, while still allowing for slight adjustments in the positions of the ME films during assembly processes. The inward curved geometry 315 offers mechanical retention without the need for additional clamping or fastening elements (e.g., one or more pins 310), simplifying the assembly processes of the medical implants 120a and 120b without causing damages to their ME films. The inward curved geometry 315 thus provides a passive yet effective means of securing the one or more ME films 110 within the hermetic enclosure 105.

The hermetic enclosure 105, in addition to the one or more ME films 110, may also incorporate additional elements and microelectronics that may be a part of the medical implants 120a and 120b. In some instances, each of the one or more ME films 110 may be housed individually in separate sections inside the hermetic enclosure 105, while the microelectronics are placed outside the hermetic enclosure 105 and embedded within the medical implants 120a and 120b. In some other instances, the one or more ME films 110 and the associated microelectronics may be arranged in stacks within a single hermetic enclosure. In case of the stacks, certain elements such as the bias magnets, providing the magnetic field, or the desiccants, protecting against the seepage of moisture, may be shared between the one or more ME films 110. As a result, multiple ME films, placed in a proximity, can function without the need for duplicate components, utilizing the space efficiently.

In examples, where the one or more ME films 110 are boxed in separate sections inside the hermetic enclosure 105, each ME film of the one or more ME films 110 may operate independently within their own sealed environment and with their own set of supporting components. The separate sections ensure that each ME film is better protected and isolated from external environmental elements such as humidity or mechanical interference. However, each ME film the one or more ME films 110 may require individual components and microelectronics, increasing the overall size and complexity of the medical implants 120a and 120b.

In some other embodiments, the hermetic enclosure 105 may include hermetic treatments such as the parylene-C coating or the atomic layer deposition (ALD), both of which can be thin-film deposition techniques that create protective, conformal layers over the surfaces of the device. These coatings may be essential for adding a barrier that may prevent contaminants from entering the enclosure and damaging the one or more ME film 110 or other sensitive internal electronics.

To apply the parylene-C coating or atomic layer deposition (ALD) coatings effectively to the interior of a hermetic enclosure, openings such as holes, gaps, or vents may be incorporated into the hermetic enclosure. The openings may allow the use of coating gases in the deposition process to permeate the inside of the hermetic enclosure, allowing protective coating to reach all surfaces including otherwise difficult to reach parts of a medical device. Without these intentional openings, it may become difficult for the coating gases to uniformly coat the internal components, leaving various areas exposed to the environment, and this may compromise the hermetic enclosure. After the coating process is completed, the holes, gaps, or vents can be filled during subsequent processing steps. This results in an airtight enclosure, ensuring a long term hermeticity in the future. By closing the openings that are used for gas permeation, the enclosure can be made completely airtight, preventing any external contaminants from entering the medical device and thus avoiding serious damage to the medical device. This two-step process—first allowing the coating gases to reach the interior, and then sealing the openings—ensures a robust and reliable hermetic seal. This design of the hermetic seal not only provides the most effective coating but also extends its life significantly. By incorporating temporary holes or vents for the coating process and then sealing them afterward, the device achieves the desired level of hermetic protection while ensuring that all internal components are thoroughly coated and shielded from a potential damage.

In some embodiments, the hermetic enclosure 105 can be designed to serve as a protective housing, which can absorb, dissipate, or redirect heat away from the one or more ME films 110 once it is exposed to high temperatures during the assembly processes. To protect the one or more ME films 101, advanced materials with superior thermal management properties, can be used within the hermetic enclosure 105. Moreover, a heatsink is added to the device, which can absorb, dissipate, or redirect heat away from the ME film once it is exposed to high temperatures during the assembly processes.

The heatsink can comprise and/or be made of a material that is non-electrically conductive and/or with a high thermal conductivity. For example, the material may include or may be a ceramic or polymer. The heatsink can be configured to operate through thermal conduction, where heat flows from the hot components around sensitive areas of a device (comprising the one or more ME films 110) to the heatsink material. As a result, the instantaneous heat generated during the process of soldering in an assembly line, where temperatures can momentarily spike, the heatsink also acts like a thermal buffer and it absorbs, stores, and dissipates heat to the environment, protecting the one or more ME films 110. Consequently, the overall temperature in the surrounding environment of the one or more ME films 110 can remain within safe limits, allowing bonding processes, like soldering connections to through-glass vias (TGVs) or other components, to successfully complete without risking any thermal damage to the ME film itself.

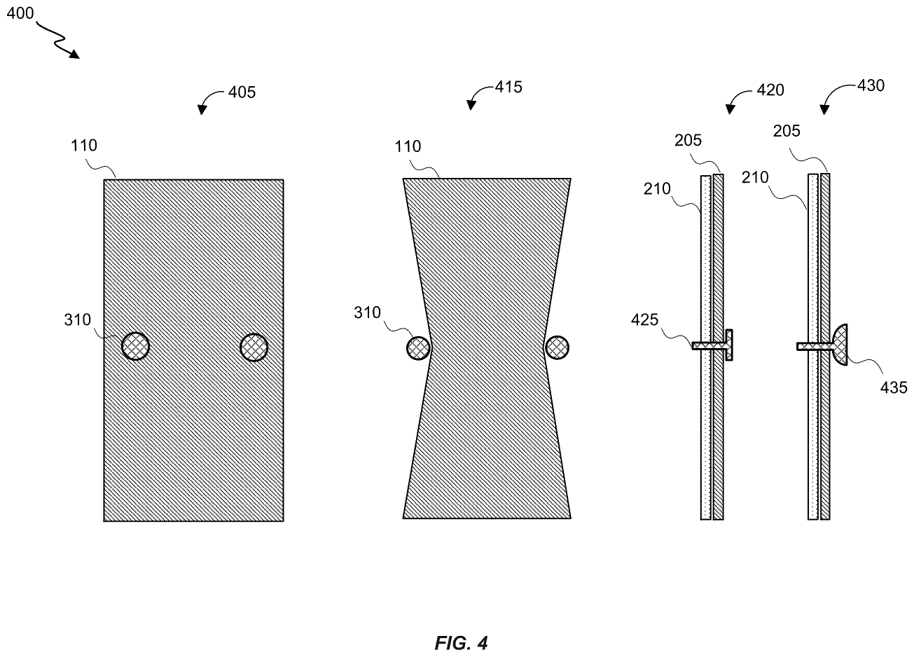

FIG. 4 illustrates one or more exemplary placements and geometric configurations of the one or more pins 310 that hold the one or more ME films 110 inside the hermetic enclosure 105. The hermetic enclosure 105 may be configured to hold varying shapes of the one or more ME films 110. The shapes of the one or more ME films 110 may include rectangular ME films 405 or trapezoidal ME films 415.

In some examples, the rectangular ME films 405 may be embedded into the hermetic enclosure 105 using the one or more pins 310, which can be configured to pierce through holes in the rectangular ME films 405. The one or more pins 310 can be positioned inside the hermetic enclosure 105 in such a way so that the pins may provide a small amount of pull or compressive forces, increasing the resonance and oscillations of the rectangular ME films 405. Moreover, the one or more pins 310 to hold the rectangular ME films 405 are located at a fixed spacing between the inner surface of the hermetic enclosure 105; as a result, the mechanical damping of the one or more ME films 110 can be minimized significantly.

In some example embodiments, only one pin from the one or more pins 310 may be placed at the midline of the one or more ME films 110, inside the hermetic enclosure 105, to reduce the pull forces that optimize the resonance of the one or more ME films 110.

In some examples, the trapezoidal ME films 415 by be used (in place of the rectangular ME films 405) that can be efficiently designed to better optimize the resonance, yet strongly hold the trapezoidal ME films 415 inside the hermetic enclosure 105. The trapezoidal ME films 415 of the one or more ME films 110 can be shaped like two trapeziums, which are joined together from each parallel side with a smaller length. The resulting shape of the trapezoidal ME films 415 may look like a rectangle that has a smaller width at the middle section compared with that of the outer sections.

The film retention features (e.g., fixation features) for the one or more ME films 110, within the hermetic enclosure 105 which may include the one or more pins 310, can be decoupled from the electrical connections, minimizing the resonance damping. The film retention features can be configured to maintain the mechanical stability of the one or more ME films 110 without interfering with the electrical properties of the one or more ME films 110.

The geometric shapes and configurations of the one or more pins 310 may affect the resonance and oscillations of the one or more ME films 110. The resonance in the one or more ME films 110 is important because it impacts the response of the one or more ME films 110 to external magnetic or electric fields. Improperly placed or significantly larger mechanical contacts can introduce mechanical damping, which results in significant energy losses during vibrations, reducing the efficiency and effectiveness of the one or more ME films 110. Therefore, the mechanical holding shapes of the one or more pins 310 may be configured to support the one or more ME films 110 without disturbing their ability to resonate freely. The film retention features may include piercing holes, slots, or notches into the one or more ME films 110 for placing the one or more pins 310 in the pierced holes, helping to fix the one or more ME films 110 within the hermetic enclosure 105. The mechanical holding elements of the hermetic enclosure 105 may be configured to have relatively low or minimum contact with the one or more ME films 110 but just enough to have them fixed in one place so that the ME films experience minimal stress or strain when excited in a magnetic or electric field.

The regions of contact of the one or more pins 310 may be identified based on a standing wave pattern induced in the one or more ME films 110 during operations. Moreover, the central portions of the one or more ME films 110, which experience the least amount of mechanical strains and small movements (relatively), is a preferred place for fixing the one or more ME films 110 using the one or more pins 310. The dimensions of mechanical contacts of the one or more pins 310 can be such that the mechanical contacts are near the center of the one or more ME films 110, covering a contact area no greater than 5-15% of the total length of the one or more ME films 110. Utilizing complex geometries such as curves, lines, or a set of point contacts may reduce the mechanical damping of the one or more ME films 110. The one or more pins 310 may feature linear or curved contact points, where the contact surface area is low or minimized to maintain the resonance properties. Linear contact geometry 420 or curved contact geometry 430 can be configured to optimize fixing of the one or more ME films 110 while maintaining their resonance characteristics. The contact surface area may be minimized either having linear contact pins 425 or curved contact pins 435, while still ensuring the mechanical stability at the fixing area of the one or more ME films 110.

In some examples, the hermetic enclosure 105, housing the one or more ME films 110, can be further constructed to apply a compressive “sandwiching” force to the one or more ME films 110. The one or more pins 310 or other fixation elements can be configured to exert compressive forces to reduce pull forces, thereby reducing the risk of delamination of the one or more ME films 110. The compressive forces maintain the structural integrity of the one or more ME films 110, while ensuring a minimal interference with the resonant behavior of the ME films, thereby enhancing the reliability and operational life of the one or more ME films 110.

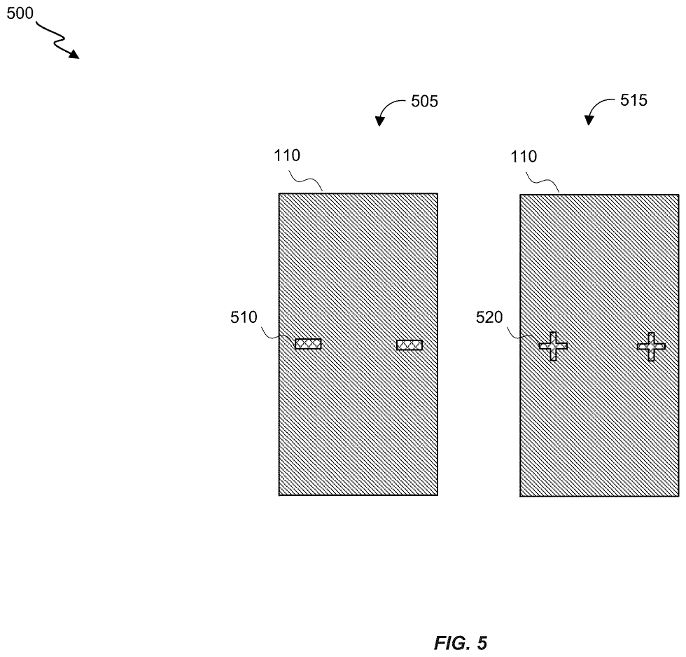

FIG. 5 shows exemplary illustrations of the varying shapes of the one or more pins 310 to hold the one or ME films 110 inside the hermetic enclosure 105. The one or more pins 310 may be rectangular pins 510 or cross-shaped pins 520. The shape of the one or more pins 310 can be determined based on the stress or strain constraints, which can be experienced by the one or more ME films 110 when they are excited by the alternating magnetic field 125. Rectangular pins 510 or cross-shaped pins 520 may be located at the contact regions of the one or more ME films 110, where they experience the least amount of stress or strain, thereby reducing their mechanical damping and preserving the resonance characteristics of the one or more ME films 110.

In some examples, the rectangular pins 510 or the cross-shaped pins 520 may be specifically designed to be fixed to the regions of the least amount of stress or strain, during the process of excitation to minimize interference with the functional properties of the one or more ME films 110. The rectangular geometry 505 and the cross-shaped geometry 515, where the size, shape, and contact area of each pin of the rectangular pins 510 or the cross-shaped pins 520 can be selected based on the mechanical behavior of the one or more ME films 110 which experience excitation by an external field. The one or more ME films 110 under excitation, generate a standing wave pattern, where the center of the one or more ME films 110 experiences the least amount of strain and small movements (relatively). The rectangular pins 510 and/or the cross-shaped pins 520 may be configured (e.g., located, positioned, oriented, etc.) to be in contact with these low-strain regions, thereby reducing the mechanical impact on the one or more ME films 110. The shapes of the one or more pins 310 may include cross-shaped, rectangular, or circular geometries, such that the size and shape may be optimized to provide sufficient mechanical support while still minimizing the contact area of the one or more pins 310 with the one or more ME films 110. Cross-shape geometry 515 also helps to position the ME film 110 at the center of the box 105, to help maintain the film 110 parallel to the box cap to prevent any physical contact with the internal surface of the box leading to mechanical damping.

The one or more pins 310 may vary in diameter or cross-sectional area also depending on the specific mechanical requirements of the hermetic enclosure 105. In someone examples, the one or more pins 310 may be configured to have circular cross-sections of varying diameter to provide mechanical stability without compromising the performance of the one or more ME films 110. Additionally, the rectangular pins 510 or the cross-shaped pins 520 may feature linear or curved contact points, wherein the contact surface area is low or minimized to maintain the resonance properties of the one or more ME films 110. The combination of contact shape, size, and location is specifically selected to ensure a stable mechanical support, while reducing the risk of mechanical damping at resonance or interference with the vibrations of the one or more ME films 110 inside the hermetic enclosure 105.

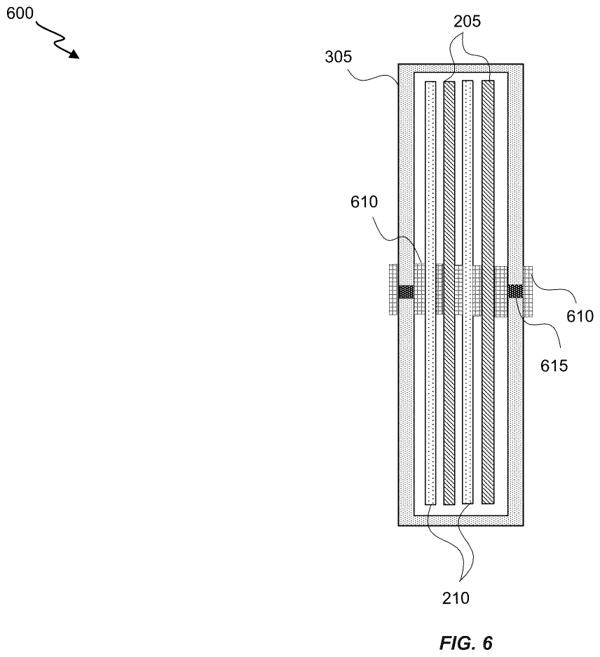

FIG. 6 shows an example illustration for placing the one or more ME films 110 inside the hermetic enclosure or film box 305 by incorporating the one or more features for various electrical connections. The electrical connections to the one or more ME films 110 may be made through a plurality of conductive elements such as metallized pads 610, through-glass-vias (TGVs) 615, microwires or other connecting media traditionally placed on the exterior surfaces of one or more ME films 110.

In some examples, where only one ME film from the one or more ME films 110 may be utilized, the metallized pads 610 may be positioned between adjacent magnetostrictive layer 205 and piezoelectric layer 210 that form a single ME film. In some other embodiments, where a plurality of ME films may be used, the metallized pads 610 may be used between two adjacent ME films to connect them, and also between adjacent magnetostrictive layer 205 and piezoelectric layer 210. Using metallized pads 610 between two adjacent ME films may reduce electrical losses, as direct electrical connections are made to the conductive elements without the need to pass through magnetostrictive layer 205 and piezoelectric layer 210.

TGVs can be typically formed by drilling small holes (vias) through a glass wafer using methods like laser drilling or photolithography and etching. The aspect ratio (the ratio of the depth to the diameter of a via) is carefully configured to ensure reliable electrical connections with a sufficient mechanical strength. Once the vias are drilled, they can be filled with a conductive material, such as copper, tungsten, or a combination of metals. The process of TGV creation may be using (for example) electroplating, chemical vapor deposition (CVD), or physical vapor deposition (PVD).

The metallized pads 610 can be located on both sides of the one or more ME films 110, ensuring a reliable electrical connection to external electronics through the TGVs 615. The TGVs 615 may be configured to penetrate the hermetic enclosure 105, and still maintaining the integrity of the seal environment inside the hermetic enclosure 105. The TGVs 615 allows the one or more ME films 110 to be connected electrically to external circuits, microelectronics or devices, with the electrical signals transmitted through the metallized pads 610, which may be positioned on both sides of the TGVs 615.

In some examples, all the components that are present inside the hermetic enclosure 105, which include the one or more ME films 110, the metallized pads 610, and the TGVs 615, may be electrically isolated by adding insulating layers between each component or element during the assembly processes. The insulating layers may prevent short circuits and unintended electrical contact between the components, ensuring the safe and reliable operation of the medical implants 120a and 120b.

Connection of the metallized pads 610 to the one or more ME films 110 may be accomplished using conductive epoxies, eutectic bonding, or compressive contacts. These methods provide robust and reliable electrical connections, ensuring that the performance of the one or more ME films 110 is maintained over time. Conductive epoxies may be used to bond the conductive elements to the one or more ME films 110, while eutectic bonding provides a durable and low-resistance connection. Alternatively, compressive contacts may be used to provide a mechanical and electrical interface between the one or more ME films 110 and the metallized pads 610 without the need for additional adhesives.

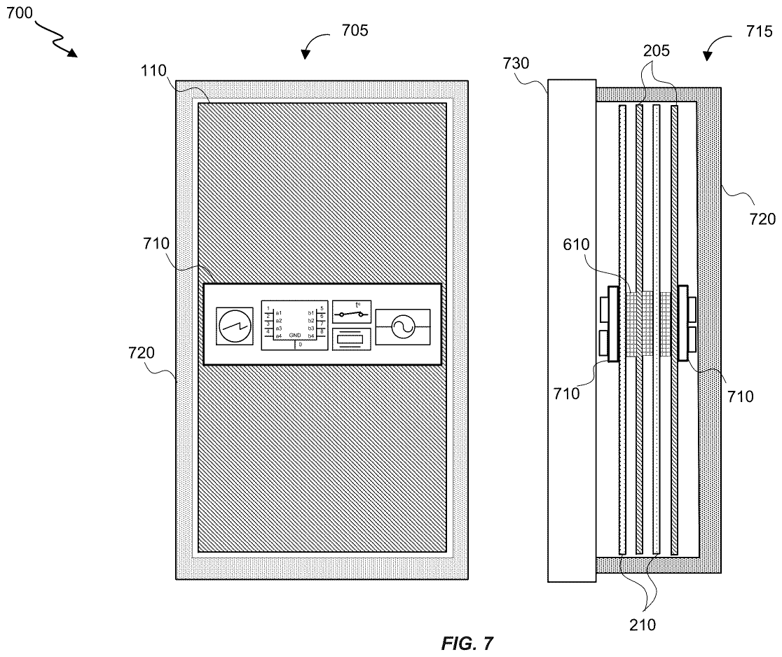

FIG. 7 shows exemplary arrangements of a plurality of microelectronics on or above the one or more ME films 110 that are on or above a carrier board 730 capped with a partial hermetic cap 720. The one or more ME films 110 can be employed as a substrate for the microelectronics 710, so that the microelectronics 710 may be positioned or packaged on a thin film structure and assembled on the carrier board 730, forming an integrated system such as a system-on-chip or system-on-module. The partial hermetic cap 720 may be configured to cover both the one or more ME films 110 and the microelectronics 710, providing protection from environmental factors while allowing for electrical connectivity. Additionally, metallized pads 610 may also be incorporated for enabling electrical connections between the one or more ME films 110.

The carrier board 730 may include a circuit board, also referred to as a breakout board or an adapter board, which is designed to interface with the microelectronics 710, smaller electronic modules, chips, or components. The carrier board 620 serves as a structural base and can be configured to carry the one or more ME films 110 and the microelectronics 710 while providing the necessary electrical connections to integrate them into larger systems or the medical implants 120a and 120b. The one or more ME films 110, functioning as a substrate, can mechanically support the microelectronics 710 and provide a foundation for packaging during the assembly processes. The combination of the one or more ME films 110, the microelectronics 710, and the carrier board 730 results in a compact and miniaturized integrated system, which can be capable of performing complex functions.

The partial hermetic cap 720, designed to provide protection to the one or more ME films 110 and the microelectronics 710, can enable routing of electrical output signals to industry-standard electronics manufacturing connections. These connections may include standard connectors, pins suitable for through-hole assembly, pads for surface-mount technology (SMT), or pads for wire-bonding. The partial hermetic cap 720 ensures that the one or more ME films 110 and microelectronics are protected from environmental factors, such as moisture and contaminants, and also provide reliable electrical connections to external electronics.

Additionally, the design of partial hermetic cap 720 may be adapted so that it becomes compatible with standard electronics manufacturing processes, such as the ones used in automated device assembly. In one example, the hermetic enclosure 105 is designed to be compatible with pick-and-place machines, which are commonly used in electronics manufacturing to place components on precise locations on circuit boards. This allows for efficient and accurate assembly of the one or more ME films 110, the microelectronics 710, and other components on the carrier board 730.

In some embodiments, the hermetic enclosure 105 and the partial hermetic cap 720 may have provisions for placing the one or more bias magnets at fixed locations. The one or more bias magnets may be positioned near or relative to the position of the one or more ME films 110.

Approaches to the placement of the one or more bias magnets within the medical implants, or sensors can affect resonance of one or more ME films 110. For example, one approach may involve placing the bias magnet outside the hermetic enclosure 105, such as in a surgical accessory as a burr hole cover. This external placement can free some internal space within the hermetic enclosure 105, enabling placing an increased number of the smaller ME films or a smaller number of larger films. By strategically positioning the bias magnet externally, the magnetic field can be directed towards the one or ME films 110. Another approach may involve positioning a central magnet to bias a group of films of the one or ME films 110. The central magnet can create a uniform magnetic field biasing the group of films of the one or ME films 110. This technique can be effective in devices where space is limited, as it reduces the use of multiple individual magnets to have a simpler design, increasing the overall magnetic flux experienced by the group of films of the one or ME films 110.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

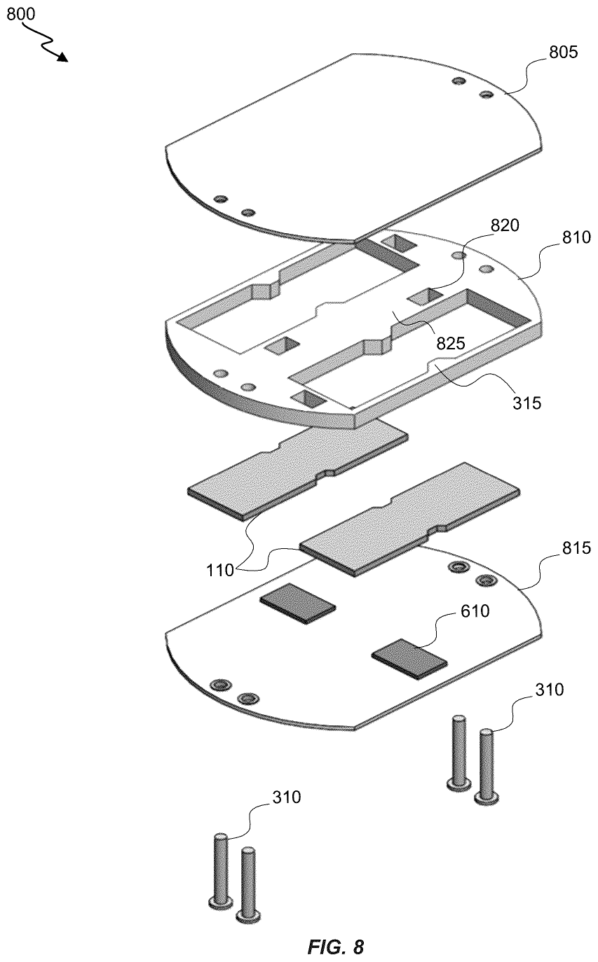

FIG. 8 shows an example illustration of an exploded view of the hermetic enclosure 105 in accordance with some embodiments of the present disclosure. The hermetic enclosure 105 may be assembled in three layers; a bottom layer 815 that can either be the carrier board or a substrate layer; a housing 810; and a lid 805. The housing 810 incorporates mechanical features that securely hold the one or more ME films 110 and provides spaces for the optimal placement of bias magnets 820. Finally, the lid 805 which may act as the uppermost covering of the hermetic enclosure 105, seals the assembly. The structure allows for the assembly of the layers through one or more pins 310, which pass through holes in the lid 805, the housing 810, and the bottom layer 815, connecting all elements during assembly.

The housing 810 may be configured with specific features to hold the one or more ME films 110 in place, including cavities to accommodate two ME side-by-side, ensuring precise positioning to avoid interference while maintaining the 5 mm minimum spacing. The bias magnets 820 can be positioned within the middle layer to provide the necessary magnetic field to activate the ME films. Additionally, the housing includes inward-curving geometry 315 that helps guide and secure the ME films within the enclosure, preventing any lateral movement. The one or more pins 310 pass through the layers, ensuring proper alignment and electrical connectivity. The design of the housing allows for a clear path for the pins to connect the films to the top and bottom surfaces, ensuring the integrity of the connections.

The bottom layer 815 may serve as the base of the hermetic enclosure 105, containing the routing circuits and the metallized pads 610 that enable the electrical connection with the ME films. The metallized pads 610 may be designed with a specific thickness to maintain sufficient clearance between the ME films and the enclosure wall. The clearance prevents any unwanted interference or contact between the ME films and the enclosure, preserving the performance of the ME films. The interior of the hermetic enclosure may provide additional space that can accommodate electronic components, such as integrated rectifier diodes and can also be used to house desiccant material, critical for preventing moisture buildup and ensuring long-term stability of the medical implants 120a and 120b.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. An apparatus comprising:
one or more magnetoelectric films, wherein a magnetoelectric film of the one or more magnetoelectric films comprises:
a magnetostrictive layer that includes a magnetostrictive element configured to be magnetized inducing a mechanical strain when an external magnetic field is applied; and
a piezoelectric layer that includes a piezoelectric element configured to generate an electrical signal in response to the mechanical strain from at least one layer of the magnetostrictive element; and
a hermetic enclosure that encloses the one or more magnetoelectric films with a spacing around the one or more magnetoelectric films wherein the spacing enables the one or more magnetoelectric films to resonate freely, wherein the hermetic enclosure is configured to mechanically hold the one or more magnetoelectric films by including one or more retention features.

2. The apparatus of claim 1, wherein the one or more retention features include:
a mechanical clamping feature configured to clamp two parallel sides of the hermetic enclosure.

3. The apparatus of claim 1, wherein the one or more retention features include one or more pins.

4. The apparatus of claim 3, wherein a surface of a pin of the one or more pins does not exceed 5 to 15 percent of total length of the one or more magnetoelectric films.

5. The apparatus of claim 1, wherein the one or more magnetoelectric films includes a plurality of magentoelectric films positioned inside the hermetic enclosure with a spacing in between two adjacent magnetoelectric films of the plurality of magnetoelectric films, wherein the spacing is filled by one or more: metallized pads, microwires or other connective media.

6. The apparatus of claim 1, wherein the one or more magnetoelectric films within the hermetic enclosure have a shape that includes two trapeziums, joined together from each parallel side having smaller length, and wherein the apparatus further includes one or more pins positioned along a longitudinal axis supporting the one or more magnetoelectric films at one or more sides of joined trapezoidal sections.

7. The apparatus of claim 1, wherein one or more electrical connections to the one or more magnetoelectric films enclosed within the hermetic enclosure include:
a plurality of metallized pads configured to connect each of two adjacent magnetoelectric films of the one or more magnetoelectric films; and
through-glass vias (TGVs) penetrating the hermetic enclosure, connecting the one or more magnetoelectric films from both sides of the TGVs to external electronics via metallized pads.

8. The apparatus of claim 1, further comprising one or more bias magnets.

9. The apparatus of claim 1, wherein the hermetic enclosure includes glass, ceramic or epoxy.

10. An apparatus comprising:
a carrier board;
one or more magnetoelectric films, wherein a magnetoelectric film of the one or more magnetoelectric film includes:
a magnetostrictive layer that includes a magnetostrictive element configured to be magnetized inducing a mechanical strain when an external magnetic field, is applied; and
a piezoelectric layer that includes a piezoelectric element configured to generate an electrical signal in response to the mechanical strain from at least one layer of the magnetostrictive element;
a partial hermetic cap that encloses the magnetoeletric film with a spacing around the magnetoelectric film to resonate, wherein the magnetoelectric film is directly positioned on the carrier board; and
a plurality of microelectronics positioned directly on the surface of the magentoelectric film as a system on module.

11. The apparatus of claim 10, wherein the plurality of microelectronics are positioned directly on either side of the magnetoelectric film of the one or more magnetoelectric films.

12. The apparatus of claim 10, wherein the partial hermetic cap enclosing the one or more magnetoeletric films and the plurality of microelectronics on a wafer film includes vias to enable electrical connection including connectors, pins through hole assembly, metallized pads or through glass vias (TGV).

13. The apparatus of claim 10, further comprising one or more bias magnets that are positioned in proximity to the one or more magnetoelectric films.

14. The apparatus of claim 10, wherein the partial hermetic cap includes glass, ceramic or epoxy.
